(19) United States
(12) Patent Application Publication
(10) Pub. No.: US 2013/0131771 A1
(43) Pub. Date: May 23, 2013

(54) CURRENT RECYCLING IN MULTIPLE LINEAR REGULATORS

(71) Applicants: Torsten Lehmann, Earlwood (AU); Yuanyuan Yang, Kingsford (AU)

(72) Inventors: Torsten Lehmann, Earlwood (AU); Yuanyuan Yang, Kingsford (AU)

(21) Appl. No.: 13/813,421

(22) PCT Filed: Aug. 1, 2011
(86) PCT No.: PCT/AU11/00970

§ 371 Date: Jan. 30, 2013

(30) Foreign Application Priority Data
Jul. 30, 2010 (AU) 2010903426

(51) Int. Cl. A61N 1/05; H02J 4/00

(52) U.S. Cl. CPC A61N 1/0541; H02J 4/00; A61N 1/0543
USPC 607/137; 307/31; 607/116

(57) ABSTRACT

Described herein is an apparatus (30) for regulating voltages across a plurality of loads (21, 23). The apparatus comprises a first linear regulator (35) for regulating a first voltage across a first load (23) and a second linear regulator (37) for regulating a second voltage across a second load (21). The apparatus also has a current recycling node (33) provided between an output of the second linear regulator (37) and an input of the first linear regulator (35) such that, in use, a total current drawn by the apparatus is less than a sum of a current flowing in the first load and a current flowing in the second load.

### FIELD OF THE INVENTION

The invention relates to the configuration and control of systems that include multiple linear regulators to regulate supply voltages.

### BACKGROUND OF THE INVENTION

In some applications, typically where there are size restraints, it is undesirable to use switch-mode power supplies because they require relatively large components such as inductors. An option for these applications is the use of linear regulators even though linear regulators tend to have a lower power-supply efficiency.

One example of an application where size is a constraint is implantable electronic devices such as cochlear implants or implanted vision prostheses. FIG. 1 illustrates an example of an implanted system 1 with platinum electrodes 3a, 3b that stimulate one or more nerves 5 of a user. The electrodes 3a, 3b are controlled by electronics in a unit 9 that is implanted subcutaneously in the user. The implanted unit 9 may have a communication link such as the transcutaneous coupled inductors 15 to an external unit 7. The link may transfer data and power. The implanted unit 9 has an internal source of power 13 that can supply a voltage V_{DDH} above reference V_{SS}. Because it is desirable to control each electrode 3a, 3b individually, the implanted unit 9 includes two driving circuits 11a and 11b for the respective electrodes.

In addition to size limitations, implantable electronic devices such as vision prostheses, cochlear implants and other implants, have other design requirements. Implantable devices are tending towards having more electrodes. Electrodes are therefore scaled down in area, resulting in increased interface impedance between the electrodes and the tissue to be stimulated by electrical signals. Hence, it is likely that required stimulation voltages are relatively high (in the order of 10 V), while most of the implanted electronics operates at much lower voltages. Traditionally, DMOS transistors are used for high-voltage stimulating parts. These transistors require low gate-driving voltages. Hence, in addition to the high stimulation voltage, biomedical implants often have two additional power rails for efficient system operation. In some biomedical applications stimulation voltages are required that are more than twice the break-down voltages of typical low-voltage transistors. Thus, in implanted unit 9, the driving circuits 11a and 11b require low-voltage control signals (V_{DDH}-V_{SSH}) and (V_{DD}-V_{SS}). This generates a need for additional power supplies V_{DD} and V_{SSH}.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

In is an object of the present invention to substantially overcome, or at least ameliorate, one or more problems with prior art arrangements.

Described herein is an arrangement for current redeployment in linear-regulator-powered electronic systems. A circuit design with at least four power rails for current recycling in a 0.35 μm high-voltage CMOS process is also demonstrated.

According to a first aspect of the invention there is provided an apparatus for regulating voltages across a plurality of loads, the apparatus comprising: a first linear regulator for regulating a first voltage across a first load; a second linear regulator for regulating a second voltage across a second load; and a current recycling node provided between an output of the second linear regulator and an input of the first linear regulator such that, in use, a total current drawn by the apparatus is less than a sum of a current flowing in the first load and a current flowing in the second load. The second linear regulator may be configured to switch between a shunt mode and a series mode.

According to a second aspect of the invention there is provided an apparatus for regulating voltages across a plurality of loads, the apparatus comprising: a first linear regulator for regulating a first voltage across a first load; a plurality of other linear regulators for regulating respective voltages across a plurality of other loads, wherein the first linear regulator and the plurality of other linear regulators are arranged in a stacked configuration; and a current recycling node provided between an output of each one of the other linear regulators and an input of an adjacent linear regulator in the stacked configuration such that, in use, a total current drawn by the apparatus is less than a sum of the currents flowing in the first load and the plurality of other loads.

According to a further aspect of the invention, there is provided an implantable device that comprises a housing for implantation; and an apparatus for regulating voltages according to the preceding paragraphs.

The implantable device may, for example, be a cochlear implant or a vision prosthesis system.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE FIGURES

Embodiments of the invention are described below with reference to the Figures, in which:

FIG. 1 is a schematic illustration of a biomedical implant that drives implanted electrodes to stimulate adjacent tissue;

FIG. 2 shows a conventional arrangement in which two voltage regulators regulate the voltage across two loads respectively;

FIG. 3A is a schematic illustration of an arrangement in which current from a first linear regulator is redeployed in a second linear regulator;

FIG. 3B is another schematic illustration of an arrangement in which current from a first linear regulator is redeployed in a second linear regulator;

FIG. 4 is a schematic illustration of the arrangement of FIG. 3 in which the first linear regulator is configured to operate in either a shunt mode or a series mode;

FIG. 5A shows a circuit implementation of an embodiment of double stacked linear regulators;

FIG. 5B shows a circuit diagram of another embodiment of double stacked linear regulators;

FIG. 5C shows an embodiment of an auxiliary linear regulator;

FIG. 5D shows an embodiment of a band gap reference;

FIG. 5E shows an embodiment of an amplifier used for the circuit implementation shown in FIG. 5B;

FIG. 6A illustrates the operation of the first linear regulator in the circuit of FIG. 5A in a series configuration;

FIG. 6B illustrates the operation of the first linear regulator in the circuit of FIG. 5A in a shunt configuration;

FIG. 7A shows time plots of a transient simulation of the circuit of FIG. 5A;

FIG. 7B shows a time plot of the charge consumption of the circuit of FIG. 5A during the simulation shown in FIG. 7A;

FIG. 8A shows the ripple measured on supply voltages for a double stacked current recycling circuit;

FIG. 8B shows the total current measured for a double stacked current recycling circuit;

FIG. 9 is a schematic illustration of an arrangement in which current is redeployed through N linear regulators to regulate the respective voltages across N loads;

FIG. 10 is a schematic illustration of an arrangement in which current is redeployed through three linear regulators to regulate the respective voltages across three loads;

FIG. 11 is an example of a circuit in which current is redeployed through three linear regulators to regulate the respective voltages across three loads;

FIG. 12 shows an auxiliary and floating power supply circuit for use in the circuit of FIG. 11;

FIG. 13 shows simulation results from a circuit in which current is redeployed through three linear regulators to regulate the respective voltages across three loads;

FIG. 14 shows simulation results of power savings in a circuit in which current is redeployed through three linear regulators to regulate the respective voltages across three loads.

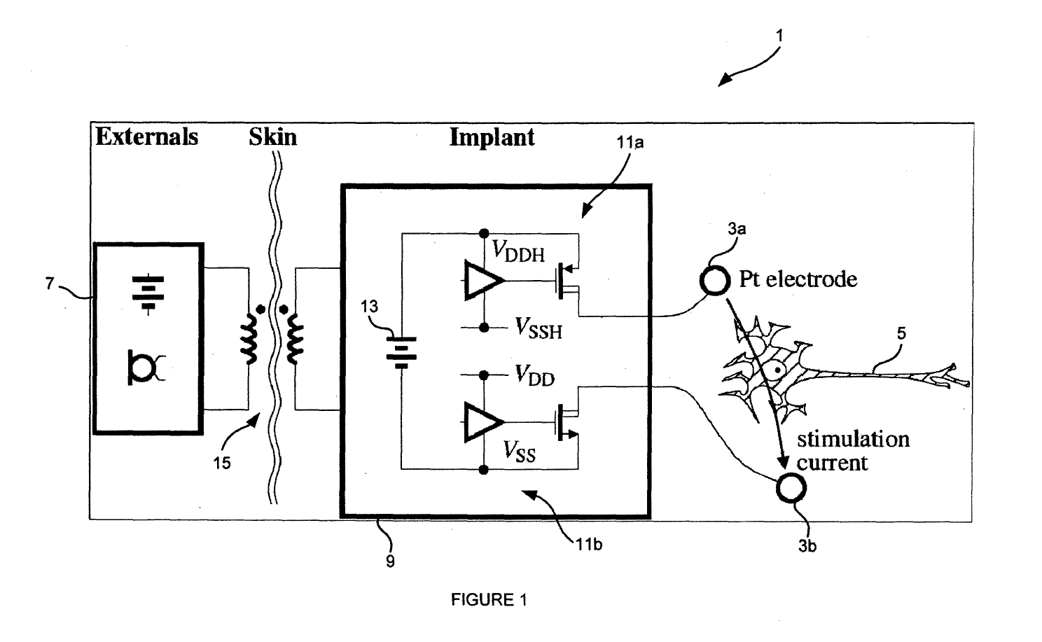

### DETAILED DESCRIPTION OF THE EMBODIMENTS

Unlike battery-powered implant electronics such as pacemakers, vision prostheses and cochlear implants often use transcutaneous coupled inductors to send data and power. The extracted and rectified electrical power is used to drive stimulating electrodes which require relatively high voltage (some 5-20 V) while modern electronic systems operate at much lower voltages. Consequently, in a traditional linear-regulator-powered electronic system, a large portion of the potential distributes on the linear regulators and ultimately the electrical energy is converted into heat.

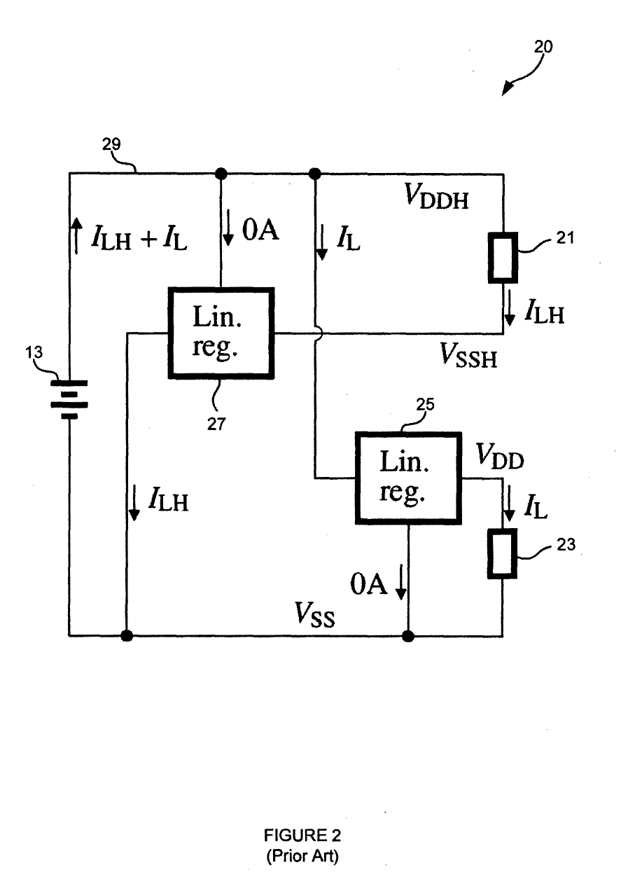

A conventional approach is illustrated in FIG. 2. System 20 has a power source 13 that provides a voltage across an upper power rail V_{DDH} and lower power rail V_{SS}. A first linear regulator 27 regulates the voltage (V_{DDH} V_{SSH}) across a first load 21. A current I_{LH} flows through load 21 to the first linear regulator 27 and thence to the lower power rail. A second linear regulator 25 regulates the voltage (V_{DD}-V_{SS}) across a second load 23. A current I_{L} flows from the second linear regulator 25 through load 23 to the lower power rail.

The total current 29 flowing from the power source 13 in the circuit 20 is (I_{LH}+I_{L}), ie the sum of the currents flowing through each of the loads 21 and 23. Note that the sum of voltages across the loads 21, 23 need not add up to the total voltage across source 13. For example, (V_{DDH}-V_{SS}) may be 10V and the voltage across each load 21, 23 may be regulated to 3V.

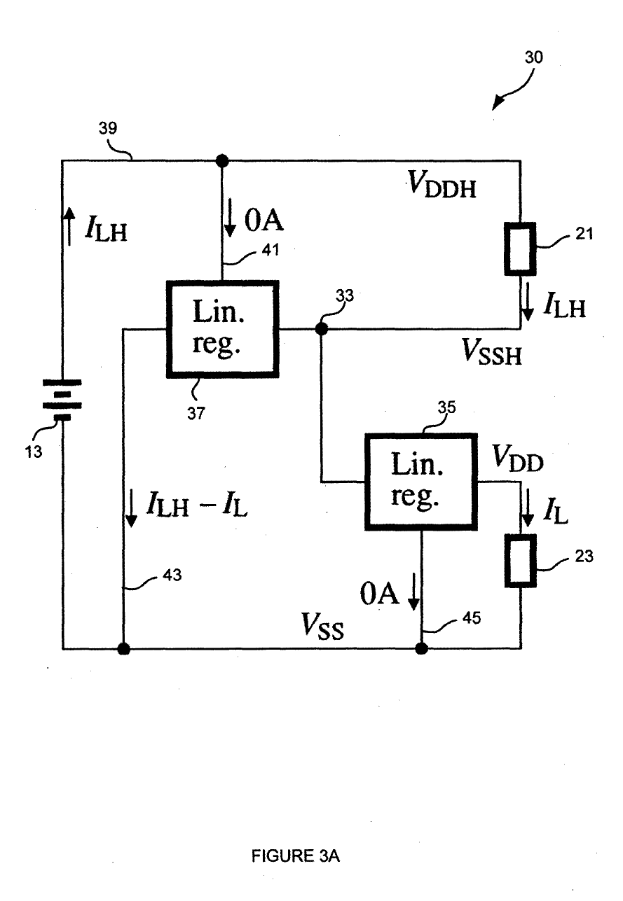

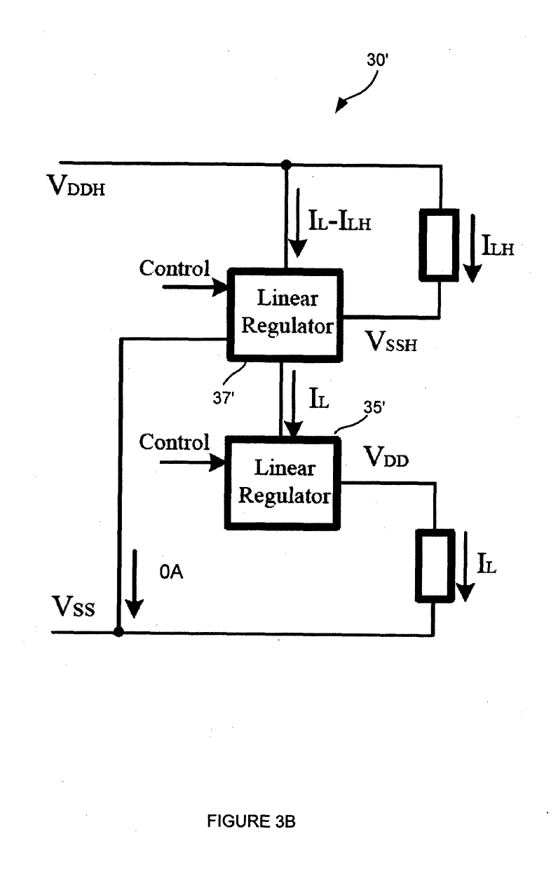

FIG. 3A shows a circuit 30 with two linear regulators 35, 37 in which a current associated with a first linear regulator 37 and its corresponding load 21 is redeployed through a second linear regulator 35 and its load 23. The circuit 30 makes better use of the high-voltage potential than the arrangement of FIG. 2 and may provide greater power-supply efficiency.

The regulators 35, 37 in circuit 30 are responsible for voltage control over the respective loads 23, 21 and for current balancing. The loads 23, 21 may, for example, be circuits for driving electrodes in a biomedical implant.

Circuit 30 has a voltage source 13 providing a potential of (V_{DDH}-V_{SS}). It is understood that the power source may be a battery, but may also be any other means by which a supply voltage can be provided to an implant such as a rectified voltage from a transcutaneous link.

There are two branches from the upper voltage rail V_{DDH}. One branch connects load 21 to the upper voltage rail V_{DDH} and the linear regulator 37. The other branch 41 connects linear regulator 37 directly to the upper voltage rail V_{DDH}. An output branch 43 of linear regulator 37 is connected to the lower voltage rail V_{SS}. Regulator 37 regulates the voltage across load 21 to (V_{DDH}-V_{SSH}).

The current flowing through load 21 is designated I_{LH}. The current splits at node 33, from which one branch connects to linear regulator 37 and another branch connects to linear regulator 35, which regulates the voltage across load 23 to (V_{DD}-V_{SS}). This splitting of the current associated with load 21 is referred to elsewhere in this specification as the “recycling” or “redeployment” of the load current.

As shown in FIG. 3A, there are two output branches from linear regulator 35. One branch 45 connects the regulator directly to the lower power rail V_{SS}. The other branch connects load 23 to the linear regulator 35 and the lower power rail. The current flowing through load 23 is designated I_{L}.

In operation as illustrated in FIG. 3A, the current in branch 41 and also in branch 45 is the supply current for each linear regulator. This is usually negligible compared with the load current and is hence assumed to be 0 A and thus the current in branch 43 is (I_{LH}-I_{L}). The total current 39 drawn from power source 13 is I_{LH}. Thus, in contrast to the arrangement of FIG. 2, the total current consumption in circuit 30 is not the sum of currents (I_{LH}+I_{L}) through the two loads, but is rather the higher current I_{LH} of the two load currents.

FIG. 3B shows an alternative schematic representation of a configuration 30′ illustrating the recycling or redeployment of current from an upper linear regulator 37′ to a lower linear regulator 35′.

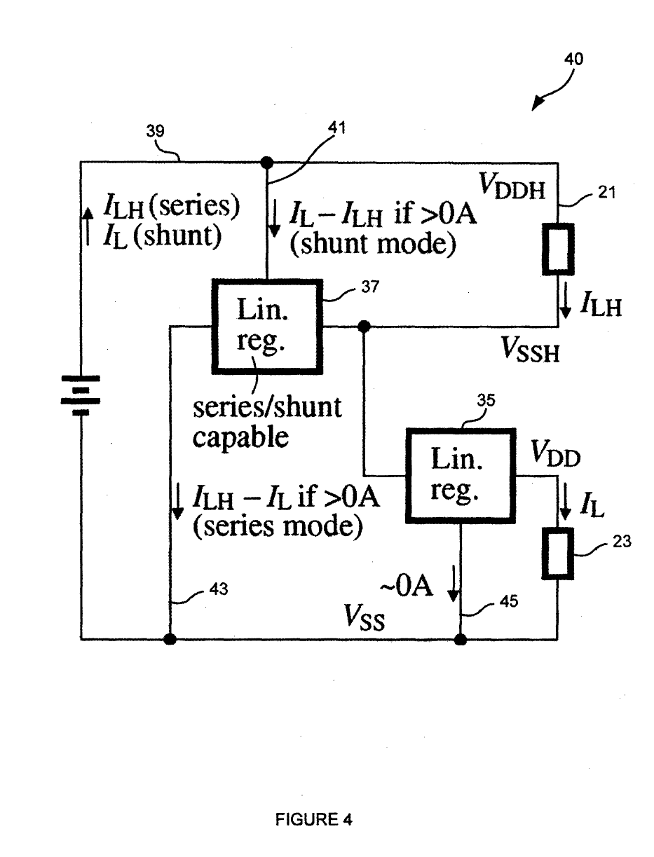

Circuit 40, as seen in FIG. 4, is the same as circuit 30 but has the feature that the upper linear regulator 37 operates in either shunt or series configuration. This serves to keep the voltage (V_{DDH}-V_{SSH}) constant even when the relative magnitudes of the two load currents vary. If I_{LH} is greater than I_{L}, linear regulator 37 operates in series mode and the current flowing in branch 43 is (I_{LH}-I_{L}). If, however, I_{L} is greater than I_{LH}, linear regulator 37 operates in shunt mode and the current flowing in branch 41 is (I_{L}-I_{LH}).

The total current 39 drawn by circuit 40 is the higher of I_{L} and I_{LH}.

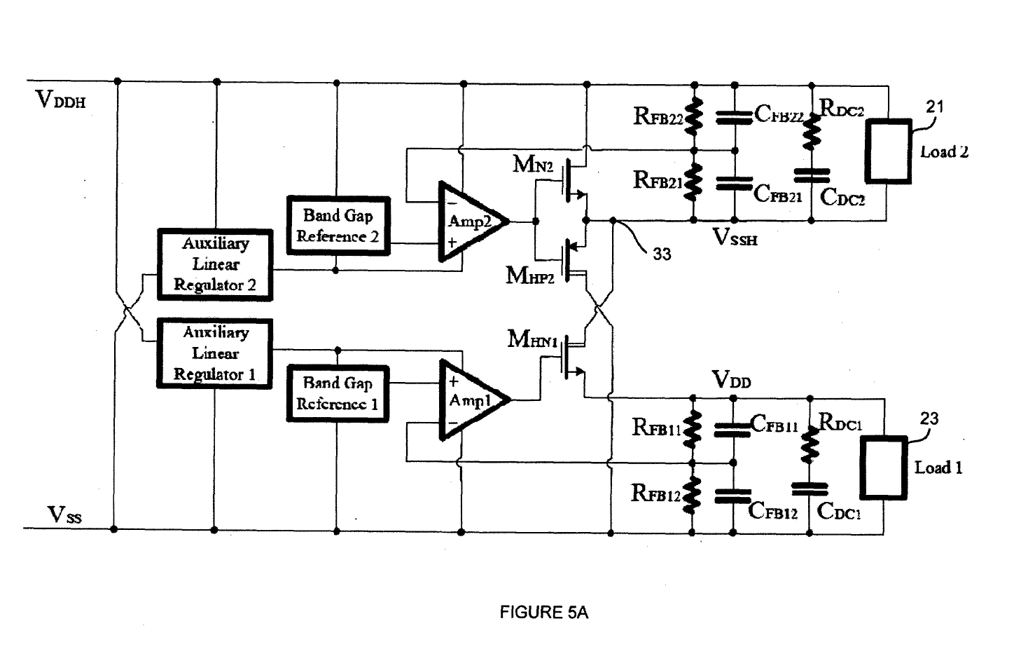

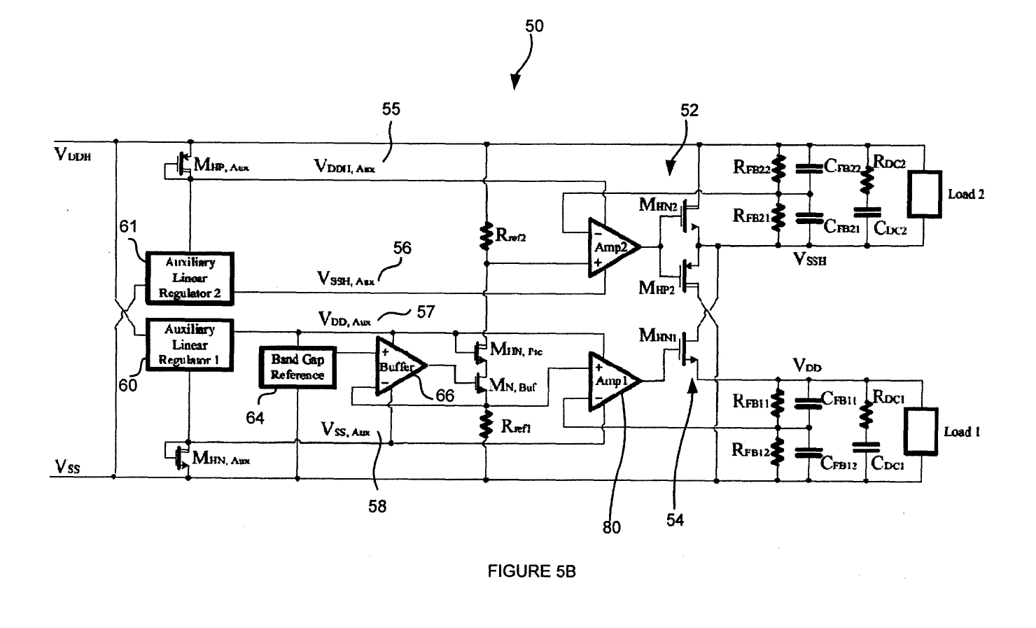

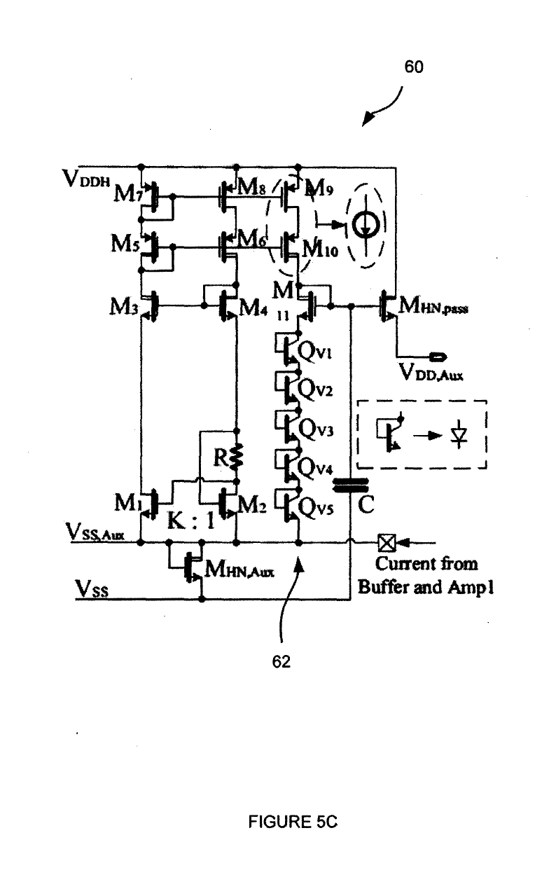

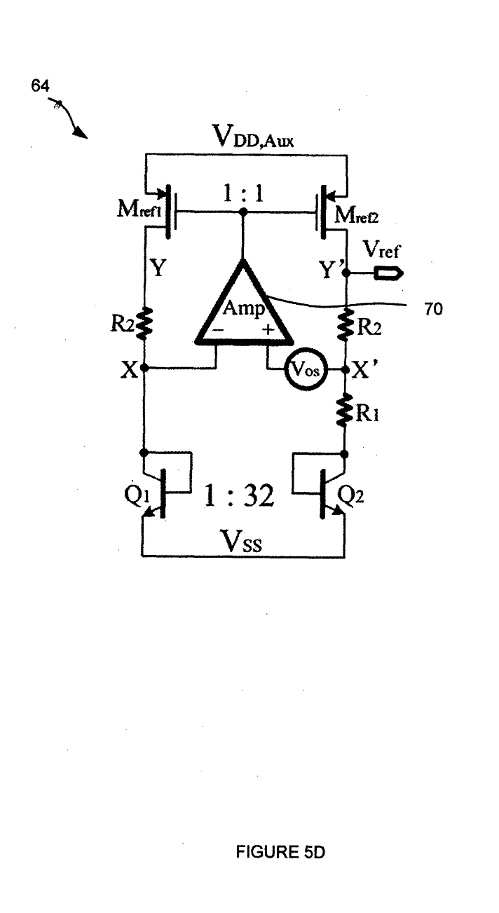

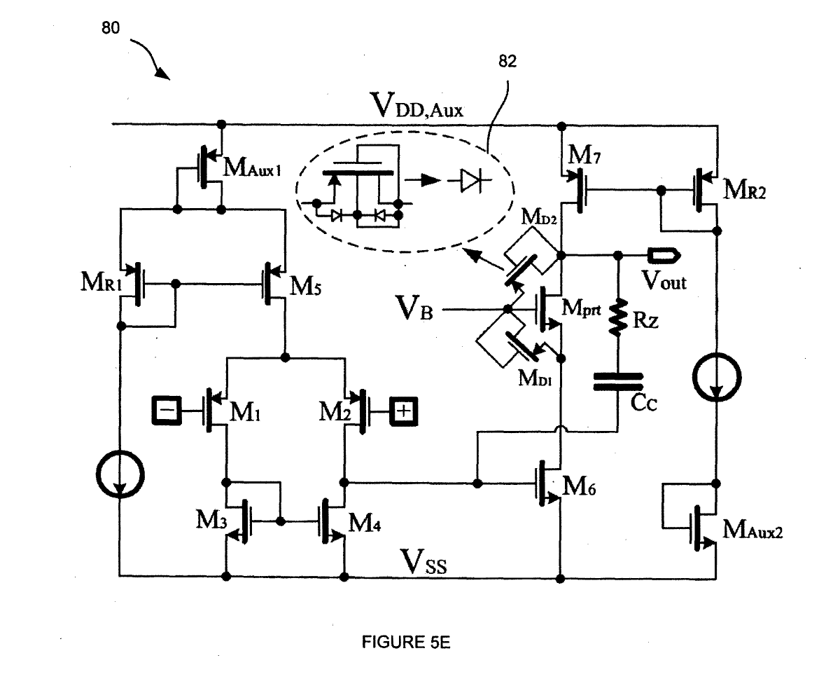

The bottom linear regulator 35 is in a conventional series configuration which provides a stable power supply for the load 23. If the current flow in the two loads is different, extra current is drawn directly from the V_{DDH} power rail through the upper linear regulator 37 (in shunt mode) when bottom load 23 uses more current than the upper load 21; when the upper load 21 uses the most current, the additional current is discharged to the V_{SS} power rail through upper linear regulator 37 (in series mode). There is no direct current path from the V_{DDH} power rail through upper linear regulator 37 to the V_{SS} power rail. The total amount of current saved in circuit 40 compared with the current consumed in circuit 20 is the smaller one of the current consumed by the two loads. By regulating the voltages on the loads, it is thus possible that high-voltage components are only needed for the linear-regulator pass elements and normal transistors can be used for electronic systems. The pass elements referred to here are, for example, the MOSFET transistors M_{N2}, M_{HP2} and K_{HN1} for the embodiment shown in FIG. 5A.

In biomedical implantable systems design, consideration is given to reliability issues like Time Dependant Dielectric Breakdown (TDDB), hot carrier degradation and junction breakdown. Voltage applied to the circuit may contribute to all three failure mechanisms, thus, for a given process, there is a tradeoff between circuit lifetime and speed, and thus at least part of the electronic system may be operated at voltages lower than maximum value specified by the foundries.

1. Double Stack Implementation: Example 1

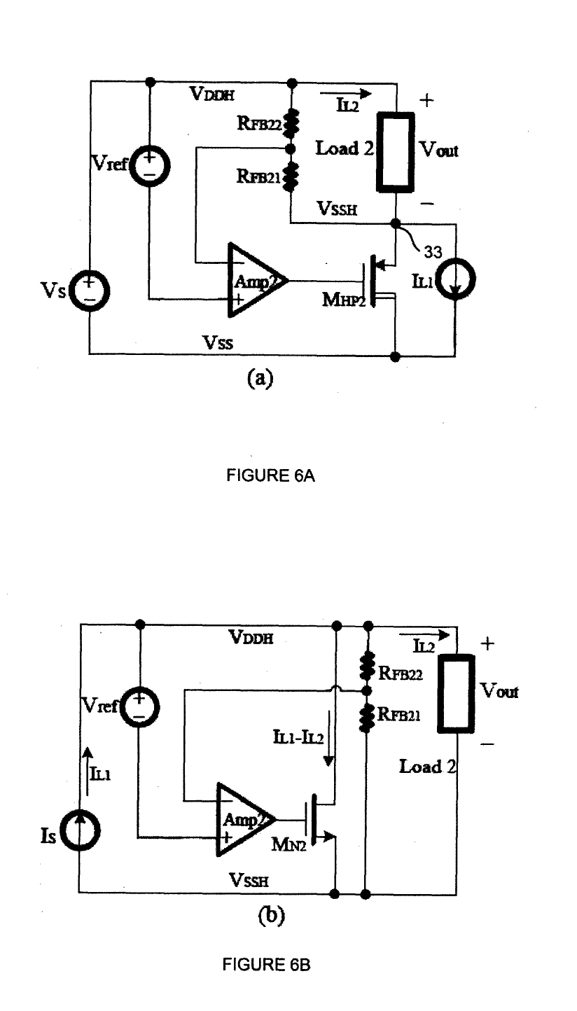

A four-power-rail current-recycling circuit corresponding to the arrangement 40 is shown in FIG. 5A. The functional circuit system is partitioned into two blocks which regulate the respective voltages across Load 1 and Load 2. The upper linear regulator 37 includes band gap reference 2, error amplifier 2 and transistors M_{N2} and M_{HP2}. The two transistors realize two regulation modes (shunt or series) according to the relative amount of current consumed by the two loads. The linear regulator 37 changes between shunt and series operation depending on which one of the transistors M_{N2} and M_{HP2} is conducting, as discussed further with reference to FIG. 6.

Auxiliary linear regulator 2 powers the control circuit for the upper linear regulator 37. Auxiliary linear regulator 2 may, for example, provide a power supply 3.3V below V_{DDH} to power the band gap reference 2 and the error amplifier 2. Band gap reference 2 generates a band gap voltage below V_{DDH}, which is supplied as one input to error amplifier 2. The other input to error amplifier 2 is a feedback signal from the output circuit components R_{FB22}, R_{FB21}, C_{FB22} and C_{FB21} connected in parallel to load 2. The output of error amplifier 2 is provided to the gates of transistors M_{N2} and M_{HP2}.

The lower linear regulator 35 includes band gap reference 1, error amplifier 1 and transistor M_{HN1}. Auxiliary linear regulator 1 powers the control circuit for the lower linear regulator 35. Auxiliary linear regulator 1 may, for example, provide a power supply 3.3V above V_{SS} to power the band gap reference 1 and the error amplifier 1. Band gap reference 1 generates a band gap voltage above V_{SS}, which is supplied as one input to error amplifier 2. The other input to error amplifier 1 is a feedback signal from the output circuit components R_{FB11}, R_{FB12}, C_{FB11} and C_{FB12} connected in parallel to load 1. The output of error amplifier 1 is provided to the gate of transistor M_{HN1}.

The sources of transistors M_{N2} and M_{HP2} are regulated at a voltage V_{SSH}. From node 33, also at this voltage, a current recycling branch connects to the drain of transistor M_{HN1}.

In the circuit of FIG. 5A the voltages across the loads can be adjusted to a suitable value for each. The extra voltage is allocated on the linear-regulator pass elements when there is an increase of power supply voltage on V_{DDH} power rail.

For compatibility in manufacturing, the circuit of FIG. 5A uses DMOS transistors with thick oxide only at the drain side. Thus, NMOS transistors in source follower configuration are used as pass elements and require relatively high voltage to switch them on.

The minimum voltage required to turn on M_{HN1} is expressed in equation (1), which is provided in Appendix A of this specification. Due to body effect in M_{HN1}, the minimum gate voltage can be further given by equation (2) of Appendix A. This gate voltage is the output of error amplifier 1.

For proper circuit operation, typically a 150 mV headroom voltage Vₕᵣ is necessary for the error amplifier output stage, thus, a minimum power supply voltage for the control circuit, ie the voltage provided by auxiliary linear regulator 1 is given by equation (3).

Assuming that M_{HN1} is sufficiently wide, the first term of the right side of equation (2) is zero, and we get a minimum gate voltage of 2.92 V for typical process values of our 0.35 μm CMOS process. A minimum power supply voltage of 3.07 V is derived from equation (3). This is lower than the maximum power supply voltage specified by the foundry. We assume the current consumed by the implantable system is no more than 2 mA, and the maximum power supply voltage from auxiliary linear regulator 1 is 3.3 V; thus, from equations (1), (2) and (3), the minimum aspect ratio of M_{HN1} can be expressed as equation (4).

Equation (4) indicates a minimum aspect ratio of M_{HN1} of approximately 688. However, the gain factor is smaller for high-voltage transistors than for normal ones, and so an aspect ratio of 1000 may be used for M_{HN1}.

The choice of specific voltages (eg around 3V), currents (eg around 2 mA), transistor dimensions (eg aspect ratio 1000) and technology (eg 0.35 μm CMOS) simply refers to a particular example of circuit 30. It will be appreciated that other choices may be appropriate. For example, 0.18 μm CMOS technology may be used, which has a break-down voltage of around 1.8 V.

System Operation

The function of the circuit of FIG. 5A is to provide constant power supply voltages for Load 1 and Load 2 even when the current consumed by the two loads varies. This is achieved by applying two regulation loops respectively to Load 1 and Load 2.

When Load 1 requires more current, voltage on power rail V_{DD} (ie the source of transistor M_{HN1}) will drop and the voltage fed back to Amplifier 1 will decrease, which results in a higher output voltage of Amplifier 1 and more current flows in M_{HN1} which compensates the power supply voltage on V_{DD} power rail. For a ΔI change of current in Load 1 where we assume originally the current in M_{HN1} is I_{D}, the change of Amplifier output voltage, or the gate voltage of M_{HN1}, can be expressed as equation (5).

The body effect is not considered in deriving equation (5); when the voltage change on power rail V_{DD} is small, ΔV_{DD} can be written as equation (6), where A_{V1} is the voltage gain of Amplifier 1.

The regulation loop for Load 2 has two regulating modes according to different load conditions, namely series regulation and shunt regulation, as shown in FIGS. 6A and 6B respectively. In both modes the voltage across Load 2 is regulated to (V_{DDH}-V_{SSH}).

The circuit in FIG. 6A shows the linear regulator 37 when operating in series regulation mode. This occurs when Load 2 consumes more current than Load 1 does. In the series configuration the linear regulator provides a variable resistance (ie through transistor M_{HP2}) in series with the load.

V_{ref} is the reference voltage provided by band gap reference 2 to the error amplifier 2, which also receives a feedback signal dependent on the voltage across Load 2. Current is redeployed or recycled from node 33 through the regulation circuit for Load 1.

The circuit in FIG. 6B represents the shunt regulation mode of linear regulator 37 under the circumstance when Load 2 uses less current than Load 1. Under series regulation condition, M_{HP2} conducts extra current to V_{SS} while for shunt regulation, difference of current from Load 1 to Load 2 (I_{L1}-I_{L2}) flows in M_{N2}. There is no direct path from V_{DDH} to V_{SS}, because to turn on M_{N2}, the gate voltage needs to be one threshold voltage higher than V_{SSH}; while to turn on M_{HP2}, the gate voltage needs to be one threshold voltage lower than V_{SSH}.

In the shunt configuration the linear regulator provides a variable resistance (ie through transistor M_{N2}) in parallel to the load.

Switching between the two regulation modes causes some voltage change on power rail V_{SSH}. For a worst case consideration, assume Load 2 consumes maximum current Iₘₐₓ in series regulation mode but no current flows in Load 1, while in shunt regulation mode, assume Load 1 uses maximum current Iₘₐₓ, but there is no current consumption in Load 2. The voltage change ΔV_{SSH} upon regulation mode switching can be expressed as equation (7) in appendix A, where A_{V2} is the voltage gain of Amplifier 2, and V_{thN0} and V_{thHP0} are threshold voltages of M_{N2} and M_{HP2} without body effect, respectively.

If similar analysis is performed on the voltage change on the V_{DD} power rail for the worst case consideration, from (6) we can further get the expression for ΔV_{DD} given in equation (8) of Appendix A.

A comparison of equations (7) and (8) indicates that the power supply voltage on the V_{DD} power rail is more stable than that of the V_{SSH} power rail.

Using a similar approach to that used to determine the aspect ration of equation (4), we obtain a value 2000 for the aspect ratio of M_{HP2}. Considering the fact that the maximum gate voltage of M_{N2} is a headroom voltage below V_{DDH}, and for the worst case where Load 1 consumes maximum current Iₘₐₓ while there is no current in Load 2, the minimum aspect ratio of M_{N2} can be expressed as equation (9) in appendix A.

The minimum value of aspect ratio may be taken as 21.3 in the 0.35 μm CMOS process, and in the circuit of FIG. 5A a value of 30 for M_{N2} may be used.

Simulation Results

Power consumption conditions alter frequently in implantable systems for power saving purposes. In order to simulate real circuit or system operation, resistors with large MOS transistors as switches are applied. By changing the control signal on MOS transistors, different load conditions may be achieved.

Before running a transient simulation for the whole circuit, a few AC simulations were performed for each of the regulation loops. A minimum phase margin of 70 degrees can be obtained by using a decoupling capacitor in series with a resistor, which generates a Left Half Plane (LHP) zero at 318 kHz.

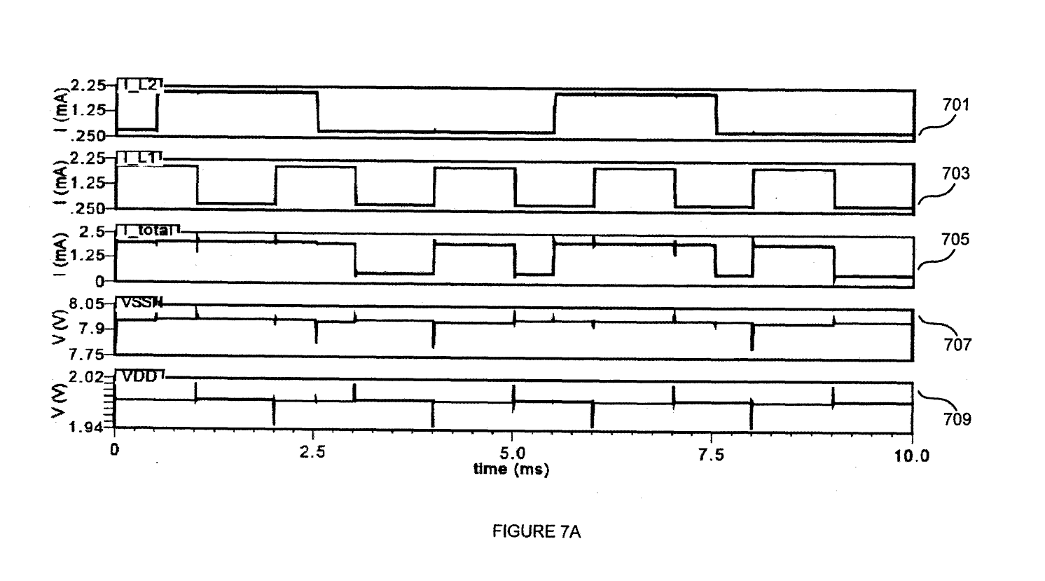

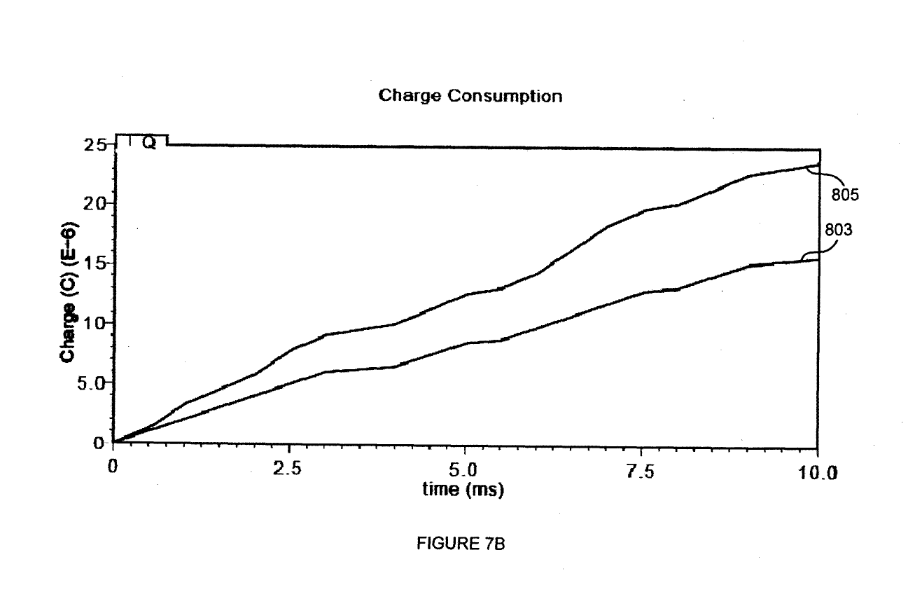

Transient simulation for different loading conditions is performed on the circuit shown in FIG. 5A, and simulated results are shown in FIG. 7A. The first two curves 701, 703 represent current in Load 2 and Load 1 respectively, where the x-axis represents time. Each load current is switched between an upper and a lower value at different frequencies.

Curve 705 shows total current consumption by the two loads. Curves 707 and 709 show voltages on power rails V_{SSH} and V_{DD} respectively. FIG. 7A indicates that current in the circuit of FIG. 5A is successfully recycled, for under all loading circumstances the total current (curve 705) is not the algebraic sum of current consumed by the two loads, but equal to the higher one of the two load currents. According to the simulation, the highest power saving factor in the loads is 49.4% and the lowest power saving factor is 24.5%, depending on the load conditions. The DC voltage change on power rail V_{DD} is about 2 mV and that on V_{SSH} power rail is 16 mV, due to finite voltage gains of the error amplifiers. However, some ripples up to 132 mV are observed on the V_{SSH} power rail when switching between series regulation mode and shunt regulation mode. This is attributed to the limited slew rate of Amplifier 2 and the bandwidth of the regulation loop.

The charge consumed by the loads can also be calculated by integrating the current, based on the transient simulation illustrated in FIG. 7A. As shown in trend 803 of FIG. 7B, the charge consumption is 15.73 μC by time 10 ms, using the current recycling technology described herein. As a comparison, the algebraic sum of charge used by the two loads is plotted as trend 805, which reaches 23.79 μC at 10 ms. This indicates an energy saving of 33.9%.

Monte Carlo simulation of both process and mismatch is also performed on the circuit of FIG. 5A. 10% variations of voltage on both V_{SSH} and V_{DD} power rails are observed, and the circuit is in stable operation under all cases. However, these variations come from discrepancy of reference voltages due to process variation and mismatch, which may be eliminated by improving the band gap reference modules.

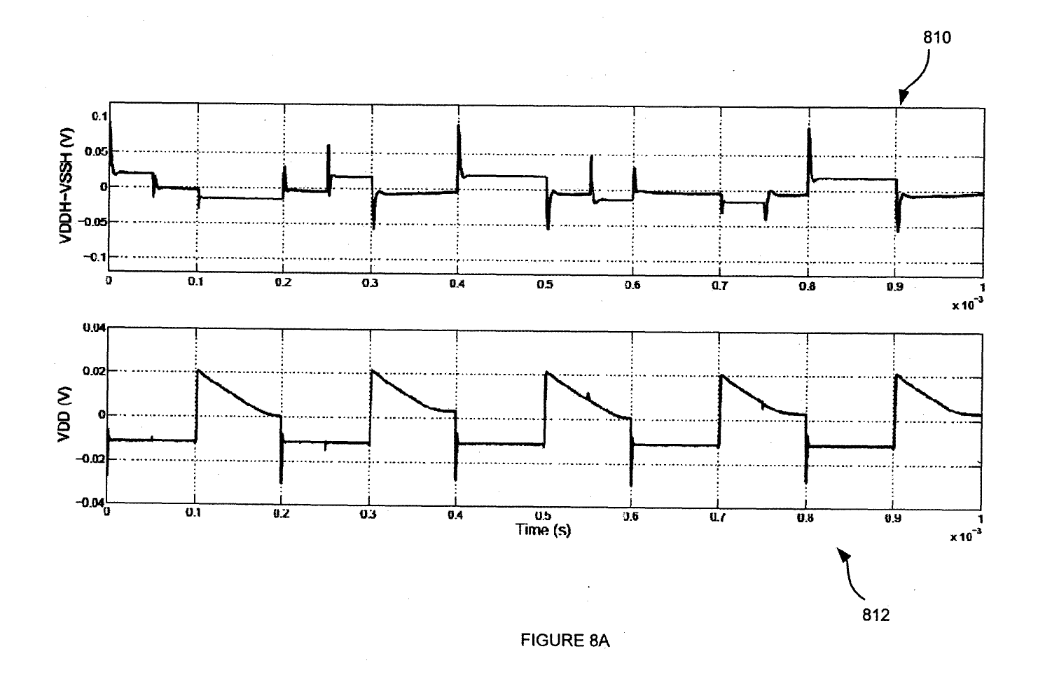

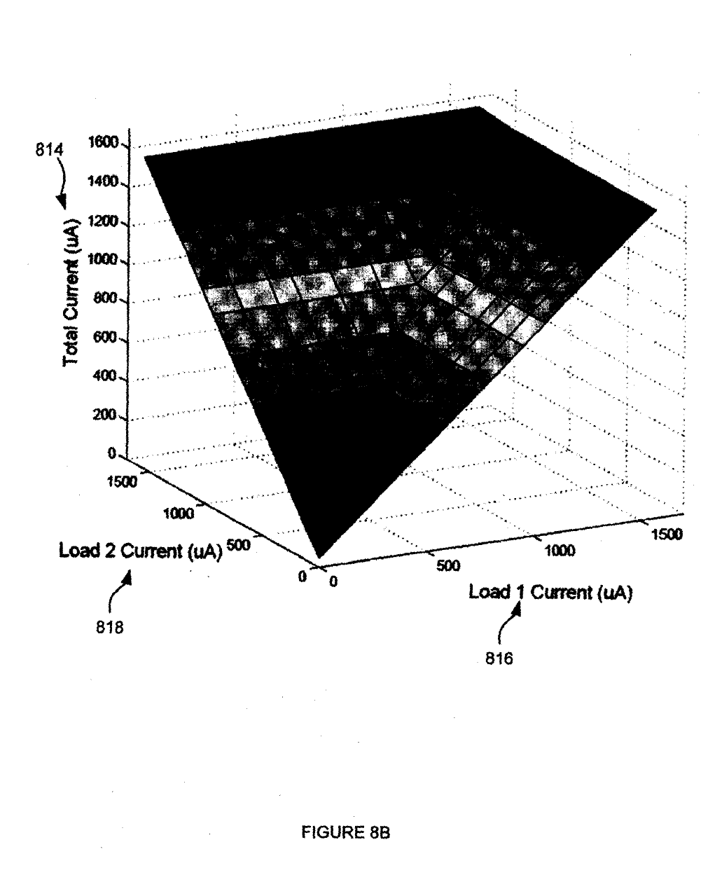

FIG. 8A shows the ripple measured on supply voltages V_{DD} 812 and (V_{DDH}-V_{SSH}) 810 for a double-stacked current-recycling circuit. FIG. 8B is a three-dimensional plot in which the x- and y-axes show the currents measured in Loads 1 and 2. The z-axis shows the measured total current of the current-recycling circuit. The plot confirms that the total current 814 does not exceed the algebraic sum of the current consumed by Load 1 816 and the current consumed by Load 2 818. The measurements were carried out on a 0.35 μm high-voltage CMOS prototype integrated circuit.

FIGS. 3 to 14 illustrate a current recycling circuit for linear regulator powered systems in a 0.35 μm high-voltage CMOS process. The described circuit generates additional power rails which can be used, for example, to power electronic systems of implants. By reusing the current, the described circuits can markedly increase power efficiency in biomedical implants like vision prosthesis and cochlear implants, compared with traditional single-linear-regulator powering schemes.

2. Double Stack Implementation: Example 2

An alternative embodiment of a current recycling circuit 50 is shown in FIG. 5B, where Load 1 and Load 2 represent electronic systems which provide electrical signals to drive neural stimulation parts. Linear regulator 52 comprises M_{HN2} and M_{HP2}. By applying different voltage signals to the gates of M_{HN2} and M_{HP2}, two regulation modes, namely series regulation and shunt regulation are achieved, providing constant power supply for Load 2. Linear regulator 54 comprises M_{HN1} which is always in series mode, providing power supply for Load 1.

Under the circumstances when the current consumptions in the two loads are different, additional current flows in M_{HP2} to V_{SS} when Load 2 uses more current than Load 1 does (series mode for Load 2). When Load 1 consumes more current than Load 2 does (shunt mode for Load 2), extra current flows in M_{HN2}. There is no direct path from V_{DDH} to V_{SS}, because to turn on M_{HN2}, the gate voltage needs to be one threshold voltage higher than V_{SSH}; while to turn on M_{HP2}, the gate voltage needs to be one threshold voltage lower than V_{SSH}.

In analogue circuits, a constant performance level can be achieved by operating circuits under a higher power supply voltage but with reduced power consumption. It is thus desirable to operate the analogue modules with supply voltages towards technology limits, for example 3.3 V for the present implementation. For efficient and reliable circuit operation, two power rails in addition to V_{DD} are required: 3 V above V_{SS} (referred to as V_{DD}) and 3 V below V_{DDH} (referred to as V_{SSH}). Thus, high gate drive voltage is required to turn on M_{HN1} and M_{HP2}.

Circuit 50 includes two auxiliary linear regulators 60, 61. FIG. 5C shows a circuit diagram for an embodiment of linear regulator 60. For safe circuit operation under all circumstances, four auxiliary power rails 55, 56, 57, 58 are used for powering the control circuit as shown in FIG. 5B. Normally, the threshold voltage of transistors in CMOS has ±0.1 V discrepancy due to process variations. Hence, in the auxiliary linear regulators 60, 61 (see FIG. 5C), desired voltages are generated by powering diode-connected Bipolar Junction Transistors (BJTs) 62 other than MOS transistors, since forward biased base-emitter junction voltages have less uncertainty than threshold voltages in MOS transistors. By properly sizing the MOS transistors and choosing the number of diode connected BJTs, we can obtain nominal voltages 0.8 V and 4 V on V_{SS,Aux} and V_{DD,Aux}, 0.8 V and 4 V below V_{DDH} on V_{DDH,Aux} and V_{SSH,Aux}.

Reference voltage in the linear power supply is generated by a single band gap reference 64, as demonstrated in FIG. 5B, and is passed down to the next stage using a buffer 66. Therefore, the reference voltage is duplicated on R_{ref2} and a band gap voltage below V_{DDH} is generated as reference voltage for Error Amplifier 2, eliminating the need for a second voltage reference. M_{HN,Ptc} is a high-voltage NMOS transistor and is used as a protection device.

FIG. 5D shows one arrangement of band gap reference 64. There are a number of factors that lead to uncertainties in reference voltage. By connecting the input of the amplifier in band gap reference to X and X′ rather than Y and Y′, as shown in FIG. 5D, the effect of mismatch in current mirror as well as in R₂ can be reduced. However, the effect of variations and mismatch in BJTs, input referred random offset in amplifier 70 and variation in R₁ cannot be eliminated. In the circuit of FIG. 5B, the last two are the dominant factors of reference voltage discrepancies. The reference voltage variations due to the random offset voltage in the amplifier can be expressed as shown in equation (10) in appendix A.

FIG. 5E shows one embodiment of a circuit for amplifier 80 of FIG. 5B. The input common mode voltage of the amplifier is a forward-biased base-emitter junction voltage, thus a two-staged amplifier with PMOS transistors as input pair and voltage shifting structure is proposed. Diode connected MOS transistors M_{Aux1} and M_{Aux2} guarantee safe power supply for the first stage of the amplifier as well as the bias circuit. Thus the output voltage can swing to V_{DD,Aux} with a headroom voltage below, which can be used as a gate control voltage for current mirror in band gap reference (eg. as shown in FIG. 5D). As the output voltage is a threshold plus an effective voltage below V_{DD,AUX}, as shown in FIG. 5E, Mₚᵣₜ is added as a cascaded transistor which works as a source follower and prevents drain voltage in M₆ from going beyond safe operation region. Auxiliary protection devices M_{D1} and M_{D2} are isolated PMOS transistors with gate, drain and body connected together, as shown in the inset 82, and they are responsible for protecting the output stage of the amplifier during power up circumstances.

As shown in FIG. 5B, M_{HN1} is an isolated high-voltage NMOS power transistor with source and body connected together. Thus, body effect can be eliminated, which reduces threshold voltage. For the same reason, M_{HP2} is a high-voltage PMOS transistor with source and body connected together. M_{HN2} is an isolated high-voltage NMOS transistor, which allows both source and body to be connected to V_{SSH}. When in series regulation mode, gate voltage is below V_{SSH}, thus gate-drain voltage potential will exceed the value for normal low-voltage transistors.

The nominal power supply voltage between the V_{DD,Aux} and V_{SS,Aux} power rails can be considered as constant. Thus for both of isolated high-voltage NMOS transistors M_{HN,Aux} and M_{HN1}, the threshold voltage discrepancies due to process variations can be cancelled out (see FIG. 5B), and the same result can be applied to M_{HP,Aux} and M_{HP2}. The nominal voltage on V_{DD} can be calculated as 2.95 V, by using nominal band gap reference voltage and feedback resistor ratio 7:5 for R_{FB11} and R_{FB12}. The variations on V_{DD} can be obtained as ±91 mV by using equations (13) and (14) shown in appendix A. Thus the maximum voltage on V_{DD} power rail, V_{DD,max} is 3.04 V.

For the worst case consideration, when maximum current Iₘₐₓ (typically less than 1.5 mA in biomedical implants), flows in M_{HN1}, the minimum aspect ratio of M_{HN1} for proper operation is given by equation (16). By using typical values, the minimum aspect ratio of M_{HN1} and be calculated as 183.6. Similar analysis can be performed on M_{HP2}) by considering the worst case that Load 2 consumes maximum current Iₘₐₓ while no current is consumed by Load 1, the minimum aspect ratio of M_{HP2} is thus calculated as 520.3. As for M_{HN2}, the value of 3.96 can also be obtained. In our design, we use 200 for M_{HN1}, and 600 for M_{HP2}, which will cover variations in gain factors. However, due to finite voltage gain A_{V2} in Error Amplifier 2, it can be predicted that there is a nominal DC voltage change on power rail V_{SSH} when regulation mode is changed as expressed in equation (17). The first term of the numerator on the right side of (17) can be reduced by using a wider M_{HN2}, thus, we use aspect ratio 50.

The performance of the system illustrated in the embodiment shown in FIG. 5B has been simulated. In the simulation, 10 V power supply on V_{DDH} is used to simulate supply voltage for neural stimulators. Resistors in series connection with large MOS transistors are used to simulate load in biomedical implantable electronics, which alter all the time for power saving purposes. Different load conditions can be achieved by providing control signals to the MOS transistors, and in the simulation it is assumed that the load consumes 1.5 mA in high power consumption mode and 500 μA in low power mode.

AC Monte Carlo simulations have been performed on both of the regulation loops, and indicate stable system operations under all load conditions as well as process variations and mismatch situations. Transient simulation demonstrates the linear regulating circuits other than Load 1 and Load 2 in FIG. 5B, including feedback resistors, consume 37.3 μA in total. Thus, when both of the loads are in high power consumption mode, the highest power saving factor as well as current efficiency can be achieved as 48.8% and 97.6% respectively. When one of the loads is in high power mode and the other in low power mode, the lowest power saving factor occurs as 23.1% and the current efficiency is 97.6% because the current is recycled. When both of the loads are in low power mode, the power saving factor is 46.3% and the current efficiency is the lowest at 93.1%.

3. Multi Stack Implementation: Example 1

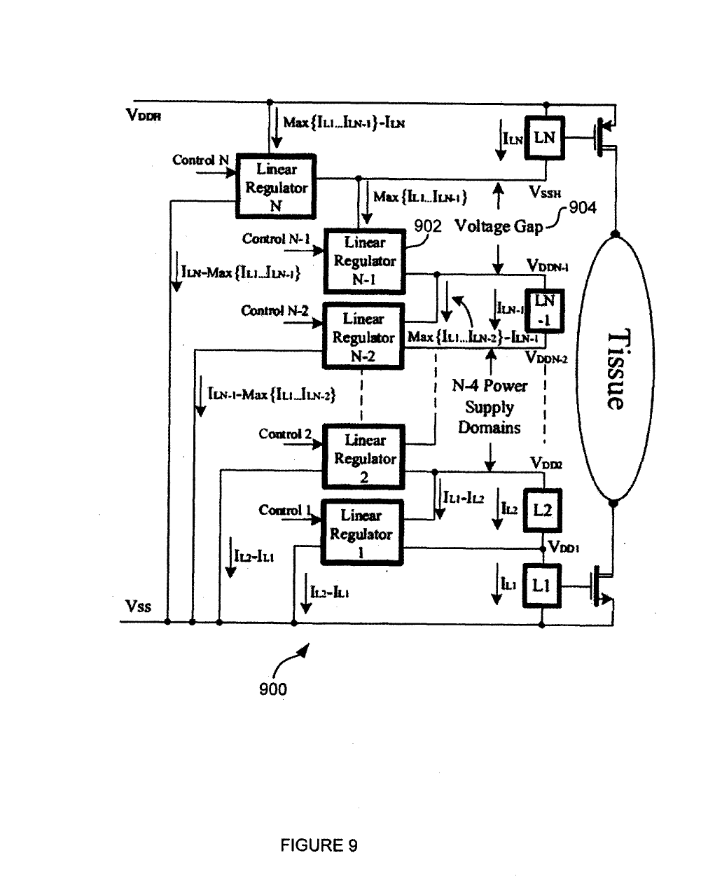

The current redeployment may also be applied in arrangements having more than two loads. FIG. 9 shows a block diagram of an embodiment of a system 900 where N linear regulators are used. The linear regulators have both series and shunt regulation capabilities with the exception of Linear Regulator (N−1) 902, which is always in series regulation mode. This ensures that the current flow through each of the loads L1 to LN is balanced and the supply voltage in each power domain is regulated. The sum of power supply voltages in all power domains is lower than the actuation supply voltage, and thus a voltage gap 904 is created between V_{SSH} and V_{DD(N-1)}. This voltage gap may be accommodated at another position in the configuration. For example instead of having the voltage gap between loads LN and LN−1, the voltage gap may be between loads L1 and L2.

Series and shunt Linear Regulator n (1≦n≦N and n≠N−1) is responsible for regulating the power supply for Load n. When the current in Load n is larger than the maximum current among Load 1 to Load (n−1), Linear Regulator n works in series regulation mode and additional current is discharged to the V_{SS} power rail. Otherwise, Linear Regulator n works in shunt regulation mode, drawing current from the V_{DD(n+1)} power rail or the V_{DDH} rail, when n=N.

Equation 18 in appendix A describes the overall current and power saving factor for this configuration. It can be seen from equation 18 that the current saving factor can be increased by having more linear regulators stacked (therefore having more low-voltage power supply domains), or distributing the load so that similar amounts of current flow through each one during operation. Also, reducing the quiescent current in linear regulators is an effective way to save power as this portion of current cannot be recycled.

Current consumption can be reduced when analogue circuits are operating at higher power supply voltages without reducing the performance of the circuit. Therefore, it is desirable to operate analogue circuits under voltages which are towards technology limits. Therefore, for the embodiment shown in FIG. 9, 10 V is used for the high-voltage actuation power supply V_{DDH} and 3 V for the electronic systems operation. This ensures sufficient headroom voltage when more than two linear regulators are stacked.

4. Multi Stack Implementation: Example 2

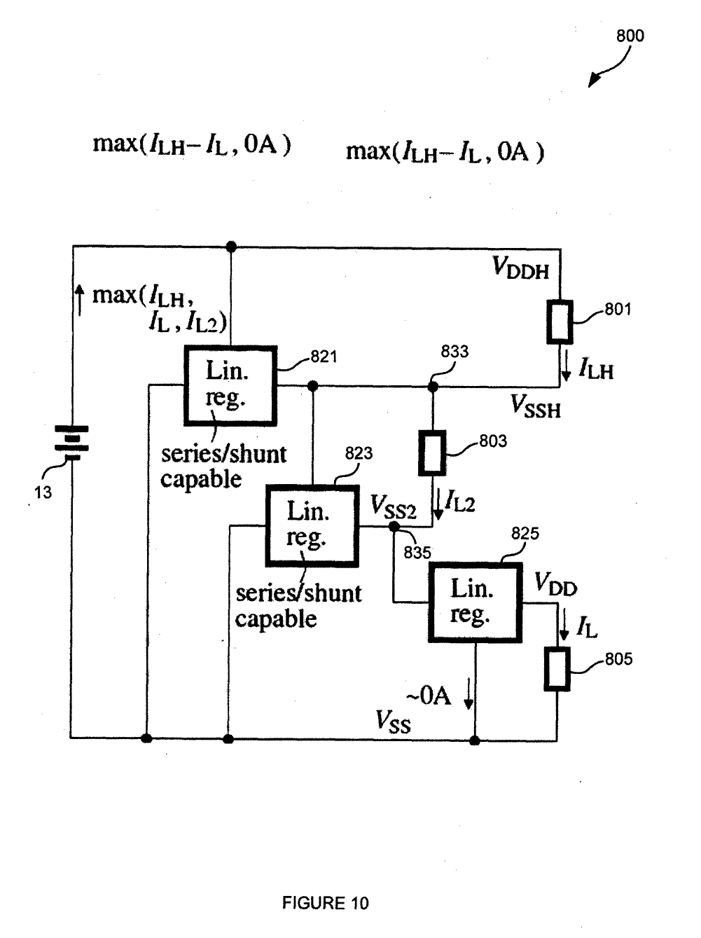

Another example of a multi stack arrangement is circuit 800, shown in FIG. 10, which has three loads 801, 803 and 805. Voltages across the loads are regulated by linear regulators 821, 823 and 825 respectively.

Circuit 800 has an upper voltage supply rail at V_{DDH} and a lower voltage supply rail at V_{SS}. Linear regulator 821 regulates the voltage across load 801 to (V_{DDH}-V_{SSH}). The current flowing through load 801 is designated I_{LH}. This current is redeployed, via node 833 to load 803.

Linear regulator 823 regulates the voltage across load 803 to (V_{SSH}-V_{SS2}). The current flowing through load 803 is designated I_{L2}. This current is redeployed, via node 835 to load 805. Linear regulator 825 regulates the voltage across load 805 to (V_{DD}-V_{SS}). The current flowing through load 805 is designated I_{L}. Linear regulator 825 may operate in a conventional series mode, but linear regulators 821 and 823 may both operate either in a shunt mode or a series mode dependent on the relative magnitudes of the load currents.

The total current drawn by circuit 800 is the maximum out of I_{LH}, I_{L} and I_{L2}. The structure of circuit 800 may be extended to applications having a greater number of loads and corresponding linear regulators.

5. Multi Stack Implementation: Example 3

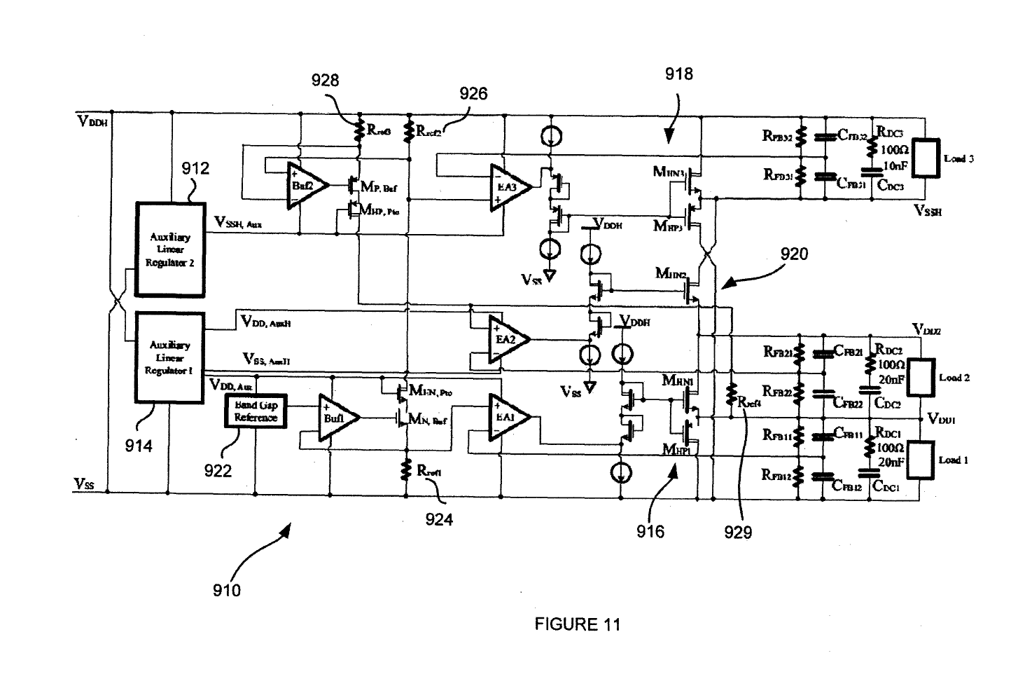

Another embodiment of a triple-stacked linear power supply circuit 910 is illustrated in FIG. 11, in which Load 1, Load 2 and Load 3 represent electronic systems, for example as used in a biomedical implantable device. For example, Load 1 and Load 3 can be used to provide gate-drive signals for the stimulation part and Load 2 can be any kind of electronics such as, for example, digital signal processor (DSP), microprocessor, analogue blocks. Two auxiliary linear regulators are used to provide power supplies for the control circuits, which operate under low-voltage supplies. Comparing with FIG. 9, power transistor pair M_{HP1} and M_{HN1} in FIG. 11 operates as Linear Regulator 1 916 with both series and shunt regulation capabilities; M_{HP3} and M_{HN3} comprise composite Linear Regulator N 918; and M_{HN2} works as Linear Regulator (N−1) 920 which is always in series regulation mode.

Three reference voltages with respect to V_{SS}, V_{DD1} and V_{DDH} are required in order to generate three regulated power supplies. A single band gap reference circuit 922 is employed and the reference voltage is duplicated as shown in FIG. 11. By using a negative feedback control circuit, a band gap reference voltage above V_{SS} and below V_{DDH} can be produced above R_{ref1} 924 and below R_{ref2} 926 respectively. The third reference voltage is generated by a second negative feedback loop which copies voltage from R_{ref2} to R_{ref3} 928 and current is injected to R_{ref4} 929 which creates a reference voltage above V_{DD1} rail.

The band gap reference voltage may suffer from discrepancies due to process variations. Thus, the power supply voltages may have uncertainties and it is expected that the voltage on the V_{DD2} power rail will have the largest variations, which can be expressed as shown in equation (19), assuming that all the factors are non-correlated.

Five high-voltage transistors are utilized in the embodiment shown in FIG. 11. M_{HP1} and M_{HN1} represent a linear regulator 916 with both series and shunt capabilities. A negative feedback loop is applied to determine the regulation mode. When Load 1 uses more current than Load 2 does, the power supply voltage on the V_{DD1} rail will drop and M_{HN1} is turned on to draw current from the V_{DD2} power rail; while Load 2 consumes more current, M_{HP1} is switched on to discharge current to V_{SS}. Linear regulator 920 comprising M_{HN2} always works in series regulation and the current flow through it is the larger current in the two loads at the bottom. Linear regulator 918 comprising M_{HP3} and M_{HN3} regulates power supply voltage on the V_{SSH} power rail with both series and shunt abilities. When current in Load 3 is larger than that flowing in M_{HN2}, M_{HP3} is turned on to conduct current to the V_{SS} power rail while in the opposite manner, M_{HN3} draws current from V_{DDH}.

There is no direct current path from the V_{DDH} to the V_{SS} power rail. Both high-voltage PMOS and NMOS transistors in the pass element pairs 916, 920 require a gate to source voltage to turn on. Thus, M_{HP3} and M_{HN3} cannot be switched on simultaneously. Likewise, when M_{HN3} is on, M_{HP1} and M_{HN1} cannot be on at the same time, preventing a direct path from V_{DDH} to V_{SS}.

Since switching the regulation mode in a pair of power transistors involves large swing of the gate drive signals, which is two threshold voltages plus two effective voltages, V_{SSH} and V_{DD1} power rails are expected to have load regulations. The worst-case voltage change due to load regulation on the V_{DD1} power rail can be expressed as shown in equation (20), by assuming regulation mode change upon the maximum current consumption in one load between Load 1 and Load 2 (an Iₘₐₓ of 1.5 mA may be used for a typical biomedical implantable device) while no current flows in the other one.

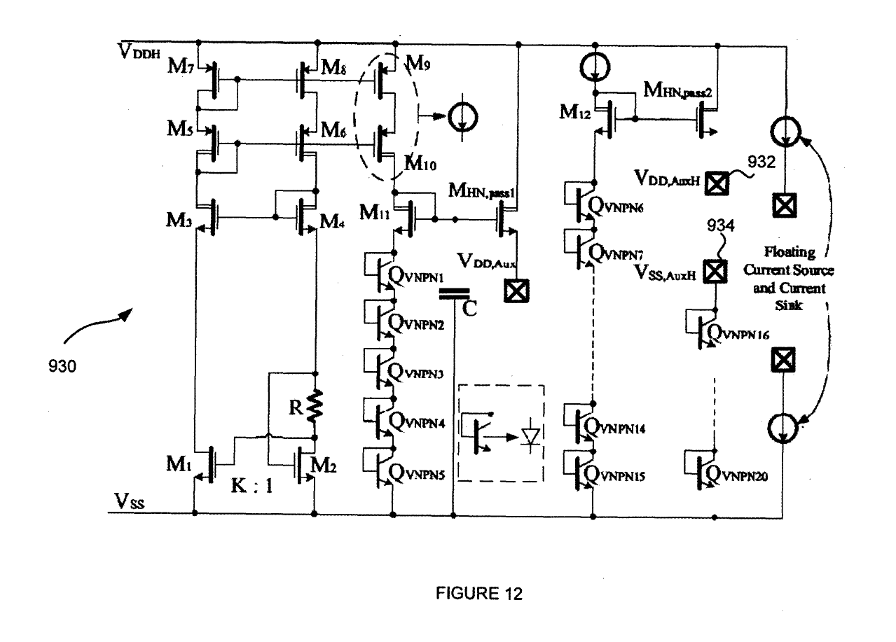

An embodiment of an auxiliary and floating power supply circuit 930 that can be used for the triple stack shown in FIG. 11 is shown in FIG. 12. Only Auxiliary Linear Regulator 1 is discussed as Auxiliary Linear Regulator 2 is the complementary implementation. As the forward biased base-emitter voltage in a Bipolar Junction Transistor (BJT) has smaller discrepancies comparing to gate to source voltage in a MOS transistor due to process variations, diode connected BJTs are used to create well-defined and reliable power supply voltages, such as V_{DD,Aux} which is 3 V above V_{SS} and V_{SSH,Aux}, which is 3 V below V_{DDH}. The gate-drive voltage for M_{HN2} is expected to be more than twice the electronic systems' supply voltages. This is well above the safe operation region of normal low-voltage transistors, thus two floating power rails V_{DD,AuxH} 932 and V_{SS,AuxH} 934 may be used to provide power supply for Error Amplifier 2. Three floating current source and current sink pairs are used to generate shifted gate drive voltages, thus the source-follower configured power transistors (M_{HN1}, M_{HN2} and M_{HP3}) can be driven by error amplifiers which are powered by 3 V low-voltage supplies.

In order to simulate electronic systems operations in a biomedical implantable device, 10 V high-voltage supply on V_{DDH} power rail is applied as actuation voltage while different power consumption conditions are achieved by modulating resistive loads powered by regulated low-voltage supplies. Two power consumption modes can be integrated into all three loads where the high current mode uses 1.5 mA and low current mode uses 500 μA, which emulates a biomedical implantable system.

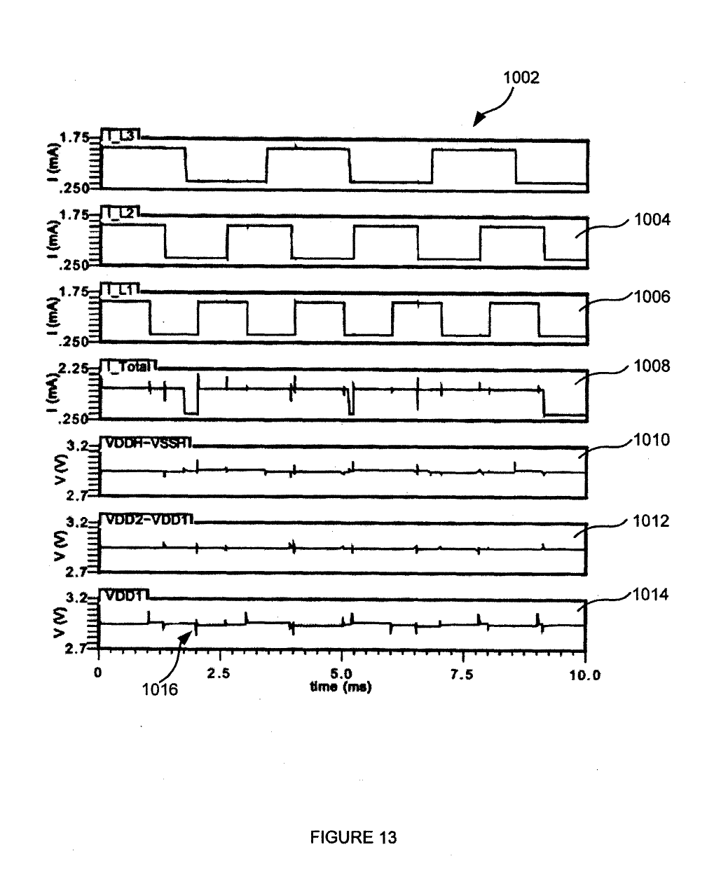

Transient simulation results are shown in FIG. 13, the first three curves 1002, 1004, 1006 representing current consumptions in the three loads. By having different periods but the same duty cycles, all loading conditions can be simulated. The fourth curve 1008 represents total current consumption and the other three 1010, 1012, 1014 are regulated power supply voltages. From the simulation results, it may be observed that the current is successfully recycled because the total current 1008 is not the algebraic sum of the current consumed by the three loads 1002, 1004, 1006, but is equal to the highest current plus the quiescent current in the control circuits (which is 50.6 μA). Therefore, the highest power saving factor may be determined as 65.5% by using equation (18), when all three loads are in high current mode, and the highest current efficiency occurs as 96.4% when any of the loads is in high current mode.

Referring to FIG. 13, some ripples 1016 up to 120 mV can be observed on the V_{SSH} and V_{DD1} 1014 power rail when switching between the two regulation modes, and this is because of limited slew rate of error amplifiers and bandwidth of the regulation loops for low power concerns. However, these ripples are acceptable since the supply voltages on the power rails do not violate safe system operation specifications.

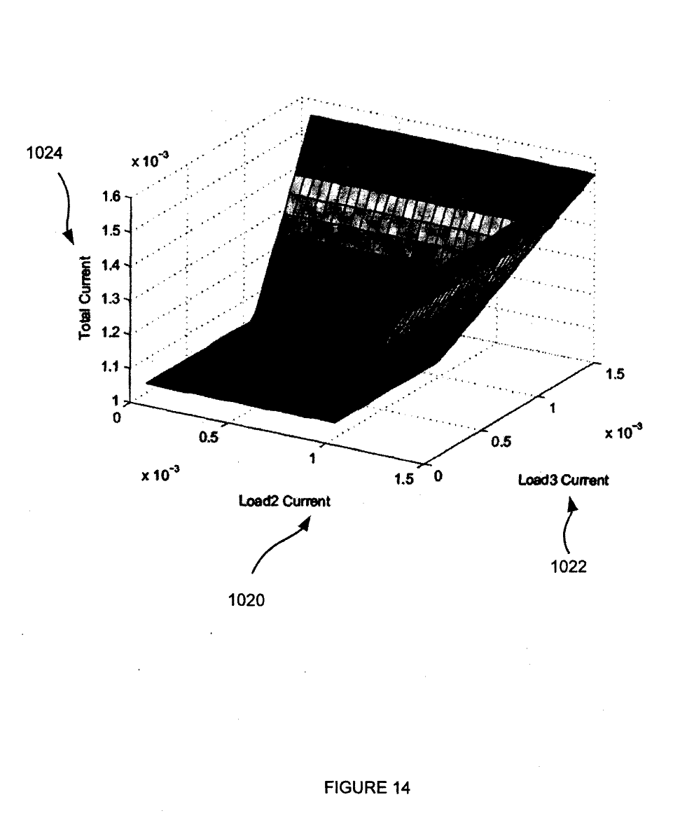

Real-time power saving effect is illustrated in FIG. 14, assuming that Load 1 consumes constant current at 1 mA while two dimensional sweeps are performed on current in Load 2 1020 and Load 3 1022. In FIG. 14, the total current consumption 1024 does not exceed the maximum current among the three loads plus the quiescent current in the control circuit.

It will be understood that the invention disclosed and defined in this specification extends to all alternative combinations of two or more of the individual features mentioned or evident from the text or drawings. All of these different combinations constitute various alternative aspects of the invention.

### APPENDIX A: Equations

Referring to the embodiment shown in FIG. 5A, the minimum voltage required to turn on M_{HN1} is given by:

$${V}_{G} = \sqrt{\frac{2 {I}_{D}}{μ {C}_{ox} \frac{W}{L}}} + {V}_{th} + {V}_{S} \qquad ( 1 )$$

where V_{G} is gate voltage of M_{HN1}, I_{D} is drain current in M_{HN1}, μCₒₓ is gain factor in M_{HN1}, W and L are width and length of M_{HN1}, Vₜₕ and V_{S} are threshold voltage and source voltage of M_{HN1} respectively.

$${V}_{G} = \sqrt{\frac{2 {I}_{D}}{μ {C}_{ox} \frac{W}{L}}} + ( {V}_{th 0} + γ ( \sqrt{\vert 2 {Φ}_{F} - {V}_{BS} \vert} - \sqrt{\vert 2 {Φ}_{F} \vert} ) ) + {V}_{S} \qquad ( 2 )$$

where Vₜₕ₀ is threshold voltage of M_{HN1} without body effect, γ is bulk effect parameter, Φ_{F} is Fermi potential, V_{BS} is body to source voltage in M_{HN1}. Other symbols are the same as in Equation (1).

V_{Aux}=V_{G}+Vₕᵣ (3)

where V_{Aux} is the voltage provided by Auxiliary linear regulator 1, V_{G} is the gate voltage of M_{HN1}, Vₕᵣ is headroom voltage in Amplifier 1.

$${( \frac{W}{L} )}_{min} = \frac{2 {I}_{max}}{μ {{C}_{ox} ( {V}_{{Aux}_{max}} - ( {V}_{th 0} + γ ( \sqrt{\vert 2 {Φ}_{F} - {V}_{BS} \vert} - \sqrt{\vert 2 {Φ}_{F} \vert} ) + {V}_{S} + {V}_{hr} ) )}^{2}} \qquad ( 4 )$$

where Iₘₐₓ is the maximum current consumed by the electronic system and V_{Auxmax} is the maximum voltage which can be provided by Auxiliary linear regulator 1.

$$Δ {V}_{G} = \frac{Δ I}{\sqrt{\frac{1}{2} μ {C}_{ox} \frac{W}{L} ( {I}_{D} + Δ I ) + \sqrt{\frac{1}{2} μ {C}_{ox} \frac{W}{L} {I}_{D}}}} - Δ {V}_{DD} \qquad ( 5 )$$

where ΔV_{G} is the gate voltage change in M_{HN1}, ΔI is current change in M_{HN1} and ΔV_{DD} is the voltage change on the V_{DD} power rail.

$$Δ {V}_{DD} = \frac{Δ I}{\begin{matrix}( {A}_{V 1} \frac{{R}_{FB 12}}{{R}_{FB 11} + {R}_{FB 12}} + 1 ) \\ ( \sqrt{\frac{1}{2} μ {C}_{ox} \frac{W}{L} ( {I}_{D} + Δ I ) + \sqrt{\frac{1}{2} μ {C}_{ox} \frac{W}{L} {I}_{D}}} )\end{matrix}} \qquad ( 6 )$$

where A_{V1} is the DC voltage gain in Amplifier 1, R_{FB11} and R_{FB12} are value of feedback resistors in FIG. 5.

$$Δ {V}_{SSH} = \frac{\begin{matrix}\sqrt{\frac{2 {I}_{max}}{{( μ {C}_{ox} \frac{W}{L} )}_{{M}_{N 2}}}} + {V}_{th N 0} + \sqrt{\frac{2 {I}_{max}}{{( μ {C}_{ox} \frac{W}{L} )}_{{M}_{HP 2}}}} + \vert {V}_{thHP 0} \vert + \\ γ ( \sqrt{\vert 2 {Φ}_{F} + {V}_{BS} \vert} - \sqrt{\vert 2 {Φ}_{F} \vert} )\end{matrix}}{{A}_{V 2} \frac{{R}_{FB 22}}{{R}_{FB 21} + {R}_{FB 22}} + 1} \qquad ( 7 )$$

where ΔV_{SSH} is the voltage change on the V_{SSH} power rail, V_{thN0} and V_{thHP0} are threshold voltages of M_{N2} and M_{HP2} respectively without body effect, V_{BS} is body-to-source voltage in M_{HP2}, A_{V2} is DC gain in Amplifier 2, R_{FBxy} terms are values of feedback resistors in FIG. 5.

$$\begin{matrix}Δ {V}_{DD} = \frac{\sqrt{\frac{2 {I}_{max}}{{( μ {C}_{ox} \frac{W}{L} )}_{{M}_{HN 1}}}}}{{A}_{V 1} \frac{{R}_{FB 12}}{{R}_{FB 11} + {R}_{FB 12}} + 1} & ( 8 ) \\ {( \frac{W}{L} )}_{min} = \frac{2 {I}_{max}}{μ {{C}_{ox} ( ( {V}_{DDH} - {V}_{SSH} ) - {V}_{thN} - {V}_{hr} )}^{2}} & ( 9 )\end{matrix}$$

where μCₒₓ is the gain factor in M_{N2}, V_{DDH} and V_{SSH} are voltages on V_{DDH} and V_{SSH} power rails, V_{thN} is threshold voltage of M_{N2}, Vₕᵣ is headroom voltage in Amplifier 2 and Imax is the maximum current consumed by the electronic system.

Referring to the embodiments described with respect to FIGS. 5B to 5E, reference voltage variations due to the random offset voltage in the amplifier can be expressed as:

$$Δ {V}_{ref 1} = {nV}_{T} ln ( 1 ∓ \frac{\vert {V}_{OS} \vert }{{nV}_{T} ln ( N )} ) ∓ \frac{{R}_{2}}{{R}_{1}} \vert {V}_{OS} \vert \qquad ( 10 )$$

where n is slope factor, V_{T} is the thermal voltage, N is the ratio between Q₂ and Q₁ and |V_{OS}| is input referred random offset in the amplifier. For small offset voltage in the amplifier, (10) can be further expressed as:

$$Δ {V}_{ref 1} = ∓ \vert {V}_{OS} \vert ( \frac{1}{ln ( N )} + \frac{{R}_{2}}{{R}_{1}} ) \qquad ( 11 )$$

From (11), it can be obtained that by using larger ratio N between Q₂ and Q₁, the reference voltage variations due the amplifier offset can be reduced. Using approximation, the discrepancies in reference voltage due to variations in R1 can be expressed as:

$$Δ {V}_{ref 2} = ∓ \frac{\vert Δ {R}_{1} \vert}{{R}_{1}} {nV}_{T} ( 1 + \frac{{R}_{2}}{{R}_{1}} ln ( N ) ) \qquad ( 12 )$$

Because of the non-correlated nature of ΔV_{ref1} and ΔV_{ref2}, the overall variations in band gap reference output can be expressed as follows, neglecting other non-dominating factors.

ΔV_{ref}=±\sqrt{|ΔV_{ref1}|²+|ΔV_{ref2}|²} (13)

The power supply voltages on V_{DD} and V_{SSH} power rails suffer from uncertainties due to the discrepancies in the reference voltage and they can be expressed as (14) and (15) respectively:

$$\begin{matrix}Δ {V}_{DD} = \pm \frac{{R}_{FB 11} + {R}_{FB 12}}{{R}_{FB 12}} \sqrt{\begin{matrix}{\vert Δ {V}_{ref} \vert}^{2} + {\vert {V}_{OS \cdot buf} \vert}^{2} + {\vert {V}_{OS \cdot amp 1} \vert}^{2} + \\ {\vert Δ {V}_{F B 1} \vert}^{2}\end{matrix}} & ( 14 ) \\ Δ {V}_{SSH} = \pm \frac{{R}_{FB 21} + {R}_{FB 22}}{{R}_{FB 22}} \sqrt{\begin{matrix}{\vert Δ {V}_{ref} \vert}^{2} + {\vert {V}_{OS \cdot buf} \vert}^{2} + {\vert Δ {V}_{r} \vert}^{2} + \\ {\vert {V}_{OS \cdot amp 2} \vert}^{2} + {\vert Δ {V}_{F B 2} \vert}^{2}\end{matrix}} & ( 15 )\end{matrix}$$

where R_{FB11}, R_{FB12}, R_{FB21}, R_{FB22} are feedback resistors as shown in FIG. 5B, V_{OS,buf}, V_{OS,amp1}, and V_{OS,amp2} are input referred random offset voltages in Buffer, Error Amplifier 1 and 2, ΔV_{FB1} and ΔV_{FB2} are error voltages in feedback resistors due to mismatch, ΔVᵣ is error voltage in R_{ref1} and R_{ref2} due to mismatch.

The minimum aspect ratio of M_{HN1} is given by:

$${( \frac{W}{L} )}_{min} = \frac{2 {I}_{max}}{{( μ {C}_{ox} )}_{HN} {( {V}_{DD , Aux} - {V}_{DD , max} - {V}_{thHN 0} - {V}_{hr} )}^{2}} \qquad ( 16 )$$

where (μCₒₓ)_{HN} is the gain factor of M_{HN1}, V_{ThHN0} is nominal threshold voltage in M_{HN1} without body effect, Vₕᵣ is headroom voltage in Error Amplifier 1.

The nominal DC voltage change on power rail V_{SSH} can be expressed as follows:

$$Δ {V}_{SSH}^{′} = \frac{\sqrt{\frac{2 {I}_{max}}{{( μ {C}_{ox} \frac{W}{L} )}_{{M}_{HN 2}}}} + {V}_{thHN 0} + \sqrt{\frac{2 {I}_{max}}{{( μ {C}_{ox} \frac{W}{L} )}_{{M}_{HP 2}}}} + \vert {V}_{thHP 0} \vert}{{A}_{V 2} \frac{{R}_{FB 22}}{{R}_{FB 21} + {R}_{FB 22}} + 1} \qquad ( 17 )$$

where V_{ThHN0} and V_{ThHP0} are threshold voltage in M_{HN2} and M_{HP2} without body effect, respectively.

Referring to the embodiments described with respect to FIGS. 10 to 14, the overall current and power saving can be expressed as:

$$S = 1 - \frac{Max \{ {I}_{L 1} , {I}_{L 2} … {I}_{LN} \} + {\sum }_{i = 1}^{N} {I}_{Qi}}{{\sum }_{i = 1}^{N} {I}_{Li}} \qquad ( 18 )$$

Where I_{Li} is the current consumption in Load i (1≦i≦N), and I_{Qi} is quiescent current in Linear Regulator i.

The variation in VDD2 can be expressed as follows:

$$Var ( {V}_{DD 2} - {V}_{DD 1} ) = \pm \frac{{R}_{FB 21} + {R}_{FB 22}}{{R}_{FB 22}} \sqrt{\begin{matrix}{\vert Δ {V}_{ref} \vert}^{2} + \\ {\sum }_{i = 1}^{2} {\vert Δ {V}_{OS , Bufi} \vert}^{2} + \\ {\sum }_{i = 1}^{3} {\vert Δ {V}_{Ri} \vert}^{2} + \\ {\vert Δ {V}_{OS , EA 2} \vert}^{2} + {\vert Δ {V}_{FB 2} \vert}^{2}\end{matrix}} \qquad ( 19 )$$

Where ΔV_{ref} is voltage variation in band gap reference output, V_{OS,Bufi} and V_{OS,EA2} are input referred offset voltages in the buffers and Error Amplifier 2, ΔV_{FB2} are error voltages in resistors R_{uref1} to R_{uref4} and feedback resistors R_{FB21}, R_{FB22} due to mismatch.

The worst-case voltage change due to load regulation on V_{DD1} power rail can be expressed as follows:

$$Δ {V}_{DD 1} = \frac{\begin{matrix}\sqrt{\frac{2 {I}_{max}}{{( μ {C}_{ox} \frac{W}{L} )}_{{M}_{HN 1}}}} + {V}_{thHN 0} + \\ \sqrt{\frac{2 {I}_{max}}{{( μ {C}_{ox} \frac{W}{L} )}_{{M}_{HP 1}}}} + \vert {V}_{thHP 0} \vert\end{matrix}}{{A}_{V 1} \frac{{R}_{FB 12}}{{R}_{FB 11} + {R}_{FB 12}} + 1} \qquad ( 20 )$$

μCₒₓ is the gain factor in the power transistors, V_{thHN0} and V_{thHP0} are threshold voltages without body effect, A_{V1} is DC gain of Error Amplifier 1. Similar result can be obtained for V_{SSH} power rail, while load regulation on V_{DD2} rail with respect to V_{DD1} rail is expected to be better than that of V_{DD1} and V_{SSH} power rails, and can be expressed as:

$$Δ ( {V}_{DD 2} - {V}_{DD 1} ) = \frac{\sqrt{\frac{2 {I}_{max}}{{( μ {C}_{ox} \frac{W}{L} )}_{{M}_{HN 2}}}}}{{A}_{V 2} \frac{{R}_{FB 22}}{{R}_{FB 21} + {R}_{FB 22}} + 1} \qquad ( 21 )$$

1. An apparatus for regulating voltages across a plurality of loads, the apparatus comprising:
a first linear regulator for regulating a first voltage across a first load;
a second linear regulator for regulating a second voltage across a second load; and
a current recycling node provided between an output of the second linear regulator and an input of the first linear regulator such that, in use, a total current drawn by the apparatus is less than a sum of a current flowing in the first load and a current flowing in the second load.

2. An apparatus according to claim 1 wherein the total current drawn by the apparatus does not exceed a maximum current flowing in any one of the plurality of loads.

3. An apparatus according to claim 1 wherein the second linear regulator is configured to switch between a shunt mode and a series mode.

4. An apparatus according to claim 3 wherein the second linear regulator is configured to operate in the shunt mode if a first current drawn by the first load is greater than a second current drawn by the second load.

5. An apparatus according to claim 3 wherein the second linear regulator comprises a transistor that provides a variable resistance path in parallel to the second load if the second linear regulator is operating in the shunt mode.

6. An apparatus according to claim 3 wherein the second linear regulator comprises a second transistor that provides a variable resistance path in series with the second load if the second linear regulator is operating in the series mode.

7. An apparatus according to claim 1 wherein the first linear regulator is configured to operate in a series mode.

8. An apparatus for regulating voltages across a plurality of loads, the apparatus comprising:
a first linear regulator for regulating a first voltage across a first load;
a plurality of other linear regulators for regulating respective voltages across a plurality of other loads, wherein the first linear regulator and the plurality of other linear regulators are arranged in a stacked configuration; and
a current recycling node provided between an output of each one of the other linear regulators and an input of an adjacent linear regulator in the stacked configuration such that, in use, a total current drawn by the apparatus is less than a sum of the currents flowing in the first load and the plurality of other loads.

9. An apparatus according to claim 8 wherein the total current drawn by the apparatus does not exceed a maximum current flowing in any one of the loads.

10. An apparatus according to claim 8 wherein the plurality of other linear regulators are configured to switch between a shunt mode and a series mode.

11. An apparatus according to claim 8 wherein the first linear regulator is configured to operate in a series mode.

12. An implantable device comprising:
a housing for implantation; and
an apparatus for regulating voltages across a plurality of loads, the apparatus comprising:
a first linear regulator for regulating a first voltage across a first load;
a second linear regulator for regulating a second voltage across a second load; and
a current recycling node provided between an output of the second linear regulator and an input of the first linear regulator such that, in use, a total current drawn by the apparatus is less than a sum of a current flowing in the first load and a current flowing in the second load.

13. An implantable device according to claim 12 comprising electrodes for stimulating tissue and driving circuits for the electrodes, wherein the apparatus regulates voltages for the driving circuits.

14. A cochlear implant comprising:
an implantable device comprising:
a housing for implantation;
an apparatus for regulating voltages across a plurality of loads, the apparatus comprising:
a first linear regulator for regulating a first voltage across a first load;
a second linear regulator for regulating a second voltage across a second load; and
a current recycling node provided between an output of the second linear regulator and an input of the first linear regulator such that, in use, a total current drawn by the apparatus is less than a sum of a current flowing in the first load and a current flowing in the second load; and
electrodes for stimulating tissue and driving circuits for the electrodes, wherein the apparatus regulates voltages for the driving circuits.

15. A vision prosthesis system comprising:
an implantable device comprising:
a housing for implantation;
an apparatus for regulating voltages across a plurality of loads, the apparatus comprising:
a first linear regulator for regulating a first voltage across a first load;
a second linear regulator for regulating a second voltage across a second load; and
a current recycling node provided between an output of the second linear regulator and an input of the first linear regulator such that, in use, a total current drawn by the apparatus is less than a sum of a current flowing in the first load and a current flowing in the second load; and
electrodes for stimulating tissue and driving circuits for the electrodes, wherein the apparatus regulates voltages for the driving circuits.

16. An implantable device comprising:
a housing for implantation; and
an apparatus for regulating voltages across a plurality of loads, the apparatus comprising:
a first linear regulator for regulating a first voltage across a first load;
a plurality of other linear regulators for regulating respective voltages across a plurality of other loads, wherein the first linear regulator and the plurality of other linear regulators are arranged in a stacked configuration; and
a current recycling node provided between an output of each one of the other linear regulators and an input of an adjacent linear regulator in the stacked configuration such that, in use, a total current drawn by the apparatus is less than a sum of the currents flowing in the first load and the plurality of other loads.
